(19) United States
(12) Patent Application Publication
(10) Pub. No.: US 2001/0007911 A1
(43) Pub. Date: Jul. 12, 2001

(54) CATALYTIC PREPARATION PROCESS OF N-ACYLGLYCINE DERIVATIVES

(76) Inventors: HOLGER GEISSLER, MAINZ (DE); SANDRA BOGDANOVIC, FRANKFURT (DE); MATTHIAS BELLER, ROSTOCK (DE); MARKUS ECKERT, MUNCHEN (DE); FRANK VOLLMULLER, MAINZ (DE)

Correspondence Address: FROMMER LAWRENCE & HAUG, 745 FIFTH AVENUE, NEW YORK, NY, 10151, US

(21) Appl. No.: 09/230,203

(22) PCT Filed: Jul. 18, 1997
(86) PCT No.: PCT/EP97/03853

§ 371 Date: None

(30) Foreign Application Priority Data
Jul. 25, 1996 (DE) 196 29 717.6

(51) Int. Cl. C07C 51/16; C07C 51/23; C07C 229/00

(52) U.S. Cl. 562/418; 562/497; 562/518; 562/519; 562/567

(57) ABSTRACT

The invention relates to a process for preparing N-acylglycine derivatives of the formula (III)

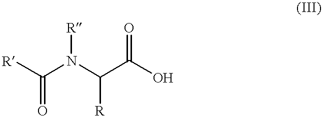

where

R is hydrogen, a carboxyl group, a saturated, straight-chain, branched or cyclic (C₁-C₁₀)alkyl radical, a monounsaturated or polyunsaturated, straight-chain, branched or cyclic (C₂-C₁₀)alkenyl radical, a (C₆-C₁₈)aryl radical, a (C₆-C₁₈)heteroaryl radical, a (C₁-C₁₀)alkyl-(C₆-C₁₈)aryl radical, a (C₁-C₁₀)alkyl-(C₆-C₁₈)heteroaryl radical or a monounsaturated or polyunsaturated (C₂-C₁₀)alkenyl-(C₆-C₁₈)aryl radical, where one or more radicals —CH₂— can be replaced by C═O or —O—,

R′ is hydrogen, a saturated, straight-chain, branched or cyclic (C₁-C₂₆)alkyl radical, a monounsaturated or polyunsaturated, straight-chain, branched or cyclic (C₂-C₂₄)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical

and

R″ is hydrogen, a saturated, straight-chain, branched or cyclic (C₁-C₂₆)alkyl radical, a monounsaturated or polyunsaturated, straight-chain, branched or cyclic (C₂-C₂₃)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical,

where R, R′ and R″ may be substituted,

which comprises carbonylating a carboxamide of the formula (II)

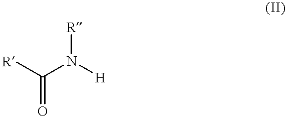

where R′ and R″ are as defined above, together with an aldehyde of the formula RCHO, where R is as defined above, in the presence of a solvent and a mixture of a palladium compound, an ionic halide and an acid as catalyst at a temperature of 20-200° C. and a CO pressure of 1-150 bar.

The present invention relates to a novel, improved process for the catalytic preparation of N-acylglycine derivatives by reacting an aldehyde with a carboxamide and carbon monoxide in the presence of a palladium compound, an ionic halide and an acid as catalyst.

Such a process, known as amidocarbonylation, which proceeds according to the reaction equation

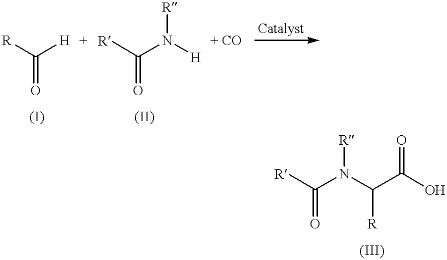

was first described by Wakamatsu et al., Chemical Communications 1971, page 1540 and in DE-A-2 115 985. The reaction was carried out in the presence of hydrogen gas at a molar ratio CO:H₂=3:1. As catalyst, cobalt carbonyl Co₂(CO)₈ was used in a concentration of 30 mmol of Co metal per liter of reaction mixture.

The same process, likewise in the presence of hydrogen gas and with additional use of a promotor compound containing a sulfoxide group, is described in EP-A-0 170 830. There, the cobalt catalyst is used in a concentration of 100 mmol of Co metal per liter of reaction mixture.

However, the comparatively large amounts of catalyst used in these processes present considerable difficulties in separating them from the fully reacted reaction mixture.

EP-B-0 338 330 describes a process for preparing N-acylglycine derivatives of the formula (III) in which R″ is hydrogen using a mixture of a palladium compound and an ionic halide as catalyst. In the process described, the palladium compound is used, calculated as palladium metal, in a concentration of 2-10 mmol per liter of reaction mixture and the ionic halide is used in an amount of 0.05-0.5 mol per liter of reaction mixture. The reaction is carried out at a pressure of 120 bar and a temperature of 120° C. The maximum yield obtained in this process was 89.9%.

DE-A-2 115 985 likewise proposes the use of a palladium-containing catalyst for amidocarbonylation. According to this document, acetaldehyde and acetamide are reacted in the presence of palladium dichloride and concentrated hydrogen chloride under CO/H₂ at a pressure of 200 bar and a temperature of 160° C., but the corresponding N-acylamino acid is obtained in a yield of only about 25%, based on the acetamide.

However, the comparatively high temperatures and pressures used here present considerable difficulties in scale-up. Likewise, they are ecologically unattractive in terms of the energy consumption. There was thus a demand for an economically improved process which gives N-acylglycine derivatives, in high yields and selectivity even with small amounts of catalyst and at relatively low pressures and temperatures.

This object is achieved by a process for preparing N-acylglycine derivatives of the formula (III)

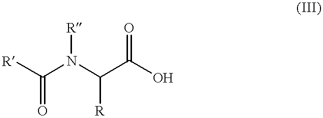

where

R is hydrogen, a carboxyl group, a saturated, straight-chain, branched or cyclic (C₁-C₁₀)alkyl radical, a monounsaturated or polyunsaturated, straight-chain, branched or cyclic (C₂-C₁₀)alkenyl radical, a (C₆-C₁₈)aryl radical, a (C₆-C₁₈)heteroaryl radical, a (C₁-C₁₀)alkyl-(C₆-C₁₈)aryl radical, a (C₁-C₁₀)alkyl-(C₆-C₁₈)heteroaryl radical or a monounsaturated or polyunsaturated (C₂-C₁₀)alkenyl-(C₆-C₁₈)aryl radical, where one or more radicals —CH₂— can be replaced by C═O or —O—,

R′ is hydrogen, a saturated, straight-chain, branched or cyclic (C₁-C₂₆)alkyl radical, a monounsaturated or polyunsaturated, straight-chain, branched or cyclic (C₂-C₂₄)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical

and

R″ is hydrogen, a saturated, straight-chain, branched or cyclic (C₁-C₂₆)alkyl radical, a monounsaturated or polyunsaturated, straight-chain, branched or cyclic (C₂-C₂₃)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical,

which comprises carbonylating a carboxamide of the formula (II)

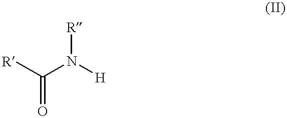

where R′ and R″ are as defined above, together with an aldehyde of the formula RCHO, where R is as defined above, in the presence of a solvent and a mixture of a palladium compound, an ionic halide and an acid as catalyst at a temperature of 20-200° C. and a CO pressure of 1-150 bar.

Preferably:

R is hydrogen, a carboxyl group, a saturated, straight-chain, branched or cyclic (C₁-C₆)alkyl radical or a monounsaturated or polyunsaturated, straight-chain, branched or cyclic (C₂-C₆)alkenyl radical, where one or more radicals —CH₂— can be replaced by C═O or —O—,

R′ is a saturated, straight-chain or branched (C₈-C₂₄)alkyl radical, in particular (C₁₀-C₁₈)alkyl radical, a monounsaturated or polyunsaturated, straight-chain or branched (C₈-C₂₄)alkenyl radical, in particular (C₁₀-C₁₈)alkenyl radical

and

R″ is hydrogen, a saturated, straight-chain or branched (C₁-C₁₂)alkyl radical, in particular (C₁-C₄)alkyl radical, or a monounsaturated or polyunsaturated, straight-chain or branched (C₂-C₈)alkenyl radical.

The radicals R, R′ and R″ may be substituted. Examples of suitable substituents are the hydroxyl group, (C₁-C₁₀)alkoxy radicals, (C₁-C₁₀)thioalkoxy radicals, di(C₁-C₁₈)alkylamino groups, (C₁-C₁₈)alkylamino groups, amino groups, protected amino groups (with Boc, Z—, Fmoc etc.), nitro groups, (C₁-C₁₀)acyloxy radicals, chloride, bromide, cyanide or fluorine.

According to the invention, the starting amides used can be any acid amides. Examples of suitable amides are formamide, acetamide, N-methylacetamide, N-isobutylacetamide, benzamide, phenylacetamide, N-butylacetamide, propionamide, butyramide, acrylamide, N-methylformamide, N-methylbenzamide, benzamide and crotonamide.

Preferred starting amides for the process of the invention are amides and N-alkylamides, in particular N-methylamides, of straight-chain or branched, saturated or unsaturated carboxylic acids having from 8 to 24 carbon atoms, for example octanoic amide, 2-ethylhexanoic amide, decanoic amide, lauramide, palmitamide, stearamide, oleamide, linolamide, linolenamide, gadoleamide and nervonic amide.

Of these, particularly preferred examples are the N-methylamides of natural fatty acids such as lauric acid, palmitic acid, stearic acid and oleic acid.

The amides of formula (II) can be used as pure substances or as mixtures. Suitable mixtures are the naturally occurring fats, e.g. coconut oil, babassu oil, palm oil, olive oil, castor oil, peanut oil, rapeseed oil, beef fat, lard or whale oil (For the composition of these fats see Fieser and Fieser, Organische Chemie, Verlag Chemie 1972, page 1208).

Any aldehydes can be used for the process of the invention. Examples of suitable alidehydes RCHO, where R is as defined above, are formaldehyde, acetaldehyde, propionaldehyde, butyraldehyde, isobutyraldehyde, furfural, crotonaldehyde, acrolein, benzaldehyde, phenylacetaldehyde, 2,4-dihydroxyphenylacetaldehyde, glyoxalic acid and α-acetoxylpropionaldehyde. It is also possible to use dialdehyde compounds. Likewise suitable are substances which can form an aldehyde under the reaction conditions specified, e.g. aldehyde oligomers such as paraformaldehyde and paraldehyde. In many cases it has been found to be useful to use formaldehyde in the form of paraformaldehyde.

The aldehyde is advantageously used in an amount of from 70 to 200 mol %, preferably from 100 to 150 mol %, based on the carboxamide.

The process of the invention is preferably carried out in one stage. The carboxamide and the aldehyde are here reacted with carbon monoxide in the presence of the catalyst to give the end product. Surprisingly, it has been found that a mixture of a palladium compound, an ionic halide and an acid is particularly effective as catalyst, so that the overall process achieves conversions of 100% of the carboxamide at selectivities of 98% to give the N-acylamino acid derivative, i.e. the yields of target product are 98%.

If desired, the process can also be carried out in two stages. In the first stage, the aldehyde and the carboxamide are reacted, with or without addition of an acid as catalyst, to form the N-acylaminomethylol of the formula (IV) which, in the second step, is reacted with carbon monoxide in the presence of a catalyst to give the end product, where the mixture of a palladium compound, an ionic halide and an acid is used in the second stage. The acid added as catalyst in the first stage is preferably the acid added as catalyst in the second stage.

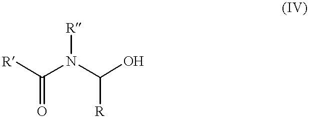

The palladium compound used can be a palladium(II) compound, a palladium(0) compound or a palladium-phosphine complex. Examples of palladium(II) compounds are palladium acetate, halides, nitrite, nitrate, carbonate, Detonates, acetylacetonate and also allylpalladium compounds. Particularly preferred representatives are PdBr₂, PdCl₂, Li₂PdBr₄, Li₂PdCl₄ and Pd(OAc)₂.

Examples of palladium(0) compounds are palladium-phosphine complexes and palladium-olefin complexes. Particularly preferred representatives are palladium-benzylidene complexes and Pd(PPh₃)₄.

In addition, when using palladium-phosphine complexes, it has been found particularly useful to use bisphosphinepalladium(II) compounds. The complexes can be used as such or can be generated in the reaction mixture from a palladium(II) compound such as PdBr₂, PdCl₂ or palladium(II) acetate with addition of phosphines such as triphenylphosphine, tritolylphosphine, bis(diphenylphosphino)ethane, 1,4-bis-(diphenylphosphino)butane or 1,3-bis(diphenylphosphino)propane. The use of phosphines having one or more chiral centers makes it possible to obtain reaction products which are enantiomerically pure or enriched with one enantiomer.

Among these palladium-phosphine complexes, particular preference is given to bis(triphenylphosphine)palladium(II) bromide—PdBr₂[PPh₃]₂—and the corresponding chloride. These complexes can be used as such or can be generated in the reaction mixture from palladium(II) bromide or chloride and triphenylphosphine.

The amount of palladium compound used is not particularly critical. However, for ecological reasons, it should be kept as small as possible. In the process of the invention, it has been found that an amount of from 0.0001 to 5 mol % of palladium compound (calculated as palladium metal), in particular 0.001-4 mol % and particularly 0.05-2 mol %, based on the carboxamide, is sufficient.

Ionic halides used can be, for example, phosphonium bromides and phosphonium iodides, e.g. tetrabutylphosphonium bromide or tetrabutylphosphonium iodide, and also ammonium, lithium, sodium and potassium bromide and iodide. Preferred halides are the bromides. The ionic halide is preferably used in an amount of from 1 to 50 mol %, in particular 2-40 mol % and very particularly 5-30 mol %, based on the carboxamide.

Acids which can be used are organic and inorganic compounds having a pKₐ<5 (relative to water). Thus, apart from organic acids such as p-toluenesulfonic acid, hexafluoropropanoic acid or trifluoroacetic acid and inorganic acids such as sulfuric acid or phosphoric acid, it is also possible to use ion-exchange resins such as Amberlyst or Nafion. Among these, particular preference is given to sulfuric acid. The acid is advantageously used in an amount of from 0.1 to 20 mol %, in particular 0.2-10 mol % and very particularly 0.5-5 mol %, based on the carboxamide.

Preferred solvents are dipolar aprotic compounds. Examples of such compounds are dioxane, tetrahydrofuran, N-methylpyrrolidone, ethylene glycol dimethyl ether, ethyl acetate, acetic acid, acetonitrile, tert-butyl methyl ether, dibutyl ether, sulfolane or N,N-dimethylacetamide or mixtures thereof. The solvents can be used in pure form or containing or saturated with product.

The N-acyl-α-amino acids obtained from the reaction can be converted into the optically pure amino acids. For the stereoselective enzymatic hydrolysis, the racemic N-acyl-α-aminocarboxylic acids obtained are usually dissolved in an aqueous reaction medium and admixed with aminoacylases, other acylases or amidases or carboxypeptidases (refs.: Enzyme Catalysis, in Organic Synthesis Ed.: K. Drauz, H. Waldmann, VCH, 1995, Vol. 1, p. 393 ff; J. P. Greenstein, M. Winitz, Chemistry of the Amino Acids; Willey, N.Y., 1961, Vol. 2, p. 1753). Depending on the specificity of the enzyme used, the reaction results in either the unprotected (L)-amino acid and the (D)-N-acylamino acid or in the (D)-amino acid and the (L)-N-acylamino acid. The optically pure N-acylamino acids can be converted by known methods either into the optically pure amino acids, e.g. by reaction with hydrochloric acid, or back into the reusable racemic N-acyl-α-aminocarboxylic acids, e.g. using acetic anhydride/glacial acetic acid or by addition of a racemase (Takeda Chemical Industries, EP- A-0 304 021; 1989).

The reaction is generally carried out at pressures of from 1 to 150 bar, preferably from 20 to 100 bar, and at temperatures of from 20 tos 200° C., preferably from 50 to 150° C.

Apart from the advantages already mentioned, for example high yield and selectivity, and a procedure which is simple to carry out in industry, the process of the invention has the further advantage that no addition of hydrogen is required.

The following examples illustrate the invention without restricting it to them.

### EXAMPLES

### Example 1

### Comparative Example

2.2 g of isovaleraldehyde, 1.5 g of acetamide, 25 ml of N-methylpyrrolidone, 0.092 g of bis(triphenylphosphine)palladium(II) chloride and 0.76 g of lithium bromide are reacted at 120 bar and 120° C. in a 300 ml autoclave. After a reaction time of 60 minutes, the mixture is analyzed by means of high-pressure liquid chromatography (HPLC). 3.9 g of N-acetylleucine are found, corresponding to a yield of 89%.

### Example 2

2.2 g of isovaleraldehyde, 1.5 g of acetamide, 25 ml of N-methylpyrrolidone, 0.017 g of palladium(II) bromide, 0.033 g of triphenylphosphine, 0.025 g of sulfuric acid and 0.76 9 of lithium bromide are reacted at 60 bar and 120° C. in a 300 ml autoclave. After a reaction time of 60 minutes, the mixture is analyzed by means of high-pressure liquid chromatography (HPLC). 4.1 g of N-acetylleucine are found, corresponding to a yield of 94%.

### Example 3

### Comparative Example

2.2 g of isovaleraldehyde, 1.5 g of acetamide, 25 ml of N-methylpyrrolidone, 0.017 g of palladium(II) bromide, 0.033 g of triphenylphosphine and 0.76 g of lithium bromide are reacted at 60 bar and 80° C. in a 300 ml autoclave. After a reaction time of 12 hours, the mixture is analyzed by means of high-pressure liquid chromatography (HPLC). 2.4 g of N-acetylleucine are found, corresponding to a yield of 55.4%.

### Example 4

2.2 g of isovaleraldehyde, 1.5 g of acetamide, 25 ml of N-methylpyrrolidone, 0.017 g of palladium(II) bromide, 0.033 g of triphenylphosphine, 0.76 g of lithium bromide and 0.025 g of sulfuric acid are reacted at 60 bar and 80° C. in a 300 ml autoclave. After a reaction time of 12 hours, the mixture is analyzed by means of high-pressure liquid chromatography (HPLC). 4.0 g of N-acetylleucine are found, corresponding to a yield of 92.4%.

### Example 5

2.2 g of isovaleraldehyde, 1.5 g of acetamide, 25 ml of N-methylpyrrolidone, 0.017 g of palladium(II) bromide, 0.76 g of lithium bromide and 0.025 g of sulfuric acid are reacted at 60 bar and 80° C. in a 300 ml autoclave. After a reaction time of 12 hours, the mixture is analyzed by means of high-pressure liquid chromatography (HPLC). 3.9 g of N-acetylleucine are found, corresponding to a yield of 89%.

### Example 6

2.2 g of isovaleraldehyde, 1.5 g of acetamide, 25 ml of N-methylpyrrolidone, 0.007 g of palladium(II) bromide, 0.014 g of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 3.25 g of N-acetylleucine are found, corresponding to a yield of 75.0%.

### Example 7

2.2 g of isovaleraldehyde, 1.5 g of acetamide, 25 ml of N-methylpyrrolidone, 0.007 g of palladium(II) bromide, 0.011 g of 1,4-bis-(diphenylphosphino)butane, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 3.5 g of N-acetylleucine are found, corresponding to a yield of 80.8%.

### Example 8

2.2 9 of isovaleraldehyde, 1.5 g of acetamide, 25 ml of N-methylpyrrolidone, 0.007 g of palladium(II) bromide, 0.011 9 of 1,4-bis-(diphenylphosphino)butane, 0.025 g of sulfuric acid and 1.31 g of sodium iodide are reacted at 60 bar and 80° C. in a 300 ml autoclave. After a reaction time of 12 hours, the mixture is analyzed by means of high-pressure liquid chromatography (HPLC). 3.6 g of N-acetylleucine are found, corresponding to a yield of 83.1%.

### Example 9

2.2 g of isovaleraldehyde, 2.2 g of butyramide, 25 ml of N-methylpyrrolidone, 0.007 g of palladium(II) bromide, 0.014 g of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 2.7 g of N-butanoylleucine are found, corresponding to a yield of 53.7%.

### Example 10

2.7 g of benzaldehyde, 1.5 g of acetamide, 25 ml of N-methylpyrrolidone, 0.007 9 of palladium(II) bromide, 0.014 g of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 3.2 g of N-acelylphenylglycine are found, corresponding to a yield of 66.2%.

### Example 11

2.2 g of isovaleraldehyde, 1.5 g of acetamide, 25 ml of dioxane, 0.007 g of palladium(II) bromide, 0.014 9 of triphenylphosphine, 0.025 g of sulfuric acid and 2.9 g of tetrabutylphosphonium bromide are reacted at 60 bar and 80° C. in a 300 ml autoclave. After a reaction time of 12 hours, the mixture is analyzed by means of high-pressure liquid chromatography (HPLC). 1.4 g of N-acetylleucine are found, corresponding to a yield of 32.3%.

### Example 12

2.2 g of isovaleraldehyde, 1.5 g of benzamide, 25 ml of N-methylpyrrolidone, 0.007 g of palladium(II) bromide, 0.014 9 of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 3.3 g of N-benzoylleucine are found, corresponding to a yield of 56.2%.

### Example 13

2.2 g of isovaleraldehyde, 1.5 g of acetamide, 25 ml of N-methylpyrrolidone, 0.017 g of palladium(II) bromide, 0.033 g of triphenylphosphine, 0.029 g of trifluoroacetic acid and 0.76 g of lithium bromide are reacted at 60 bar and 80° C. in a 300 ml autoclave. After a reaction time of 12 hours, the mixture is analyzed by means of high-pressure liquid chromatography (HPLC). 3.1 g of N-acetylleucine are found, corresponding to a yield of 71.6%.

### Example 14

2.2 g of isovaleraldehyde, 1.5 g of acetamide, 25 ml of N,N-dimethylformamide, 0.007 g of palladium(II) bromide, 0.013 g of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 1.75 g of N-acetylleucine are found, corresponding to a yield of 40.0%.

### Example 15

2.2 g of isovaleraldehyde, 1.5 g of acetamide, 25 ml of N-methylpyrrolidone, 0.029 g of tris(dibenzylideneacetone)dipalladium(0), 0.033 g of triphenylphosphine, 0.025 g of sulfuric acid and 0.76 g of lithium bromide are reacted at 60 bar and 80° C. in a 300 ml autoclave. After a reaction time of 12 hours, the mixture is analyzed by means of high-pressure liquid chromatography (HPLC). 2.62 g of N-acetylleucine are found, corresponding to a yield of 60%.

### General Procedure I for Examples 16-20

25.0 ml of a 1M N-methylpyrrolidone solution of the aldehyde and of the amide are reacted with 16.6 mg of palladium(II) bromide, 33.1 mg of triphenylphosphine, 0.76 g of lithium bromide and 25 mg of sulfuric acid at 120° C. for 12 hours under 60 bar of carbon monoxide pressure in a 300 ml autoclave. The reaction mixture was analyzed by means of high-pressure liquid chromatography (HPLC).

### Example 16

3.1 g of para-fluorobenzaldehyde and 1.5 g of acetamide were reacted using the general procedure I. 4.7 g of N-acetyl-para-methoxyphenylglycine are found, corresponding to a yield of 89%. Selected NMR data: ¹H-NMR (400 MHz, DMSO-d₆, 25° C.): δ=8.6 (d, 1H, NH), 5.3 (d, 1H, α-CH), 1.9 (s, 3H, COCH₃).

### Example 17

3.0 g of phenylacetaldehyde and 1.5 g of acetamide were reacted using the general procedure I. 2.6 g of N-acetylphenylalanine are found, corresponding to a yield of 48.3%. Selected NMR data: ¹H-NMR (400 MHz, DMSO-d₆, 25° C.): δ=8.2 (d, 1H, NH), 4.4 (dt, 1H, α-CH), 1.8 (s, 3H, COCH₃).

### Example 18

2.6 g of 3-methylthiopropionaldehyde were reacted with 1.5 g of acetamide using the general procedure I. 3.6 g of N-acetylmethionine are found, corresponding to a yield of 75.3%. Selected NMR data: ¹H-NMR (400 MHz, DMSO-₆, 25° C.): δ=8.2 (d, 1H, NH), 4.1 (dt, 1H, α-CH), 1.8 (s, 3H, COCH₃).

### Example 19

3.5 g of ortho-chlorobenzaldehyde and 1.5 g of acetamide were reacted using the general procedure I. 4.7 g of N-acetyl-ortho-chlorophenylglycine are found, corresponding to a yield of 82.6%. Selected NMR data: ¹H-NMR (400 MHz:, DMSO-d₆, 25° C.): δ=8.7 (d, 1H, NH), 5.8 (d, 1H, α-CH), 1.9 (s, 3H, COCH₃).

### Example 20

3.9 g of 2-naphthaldehyde and 1.5 g of acetamide were reacted using the general procedure I. 4.6 g of N-acetyl-2-naphthylglycine are found, corresponding to a yield of 75.7%. Selected NMR data: ¹H-NMR (400 MHz, DMSO-d₆, 25° C.): δ=8.8 (d, 1H, NH), 5.6 (d, 1H, α-CH), 2.0 (s, 3H, COCH₃).

### General Procedure II for Examples 21-28

25.0 ml of a 1M N-methylpyrrolidone solution of the aldehyde and of the amide are reacted with 16.6 mg of palladium(II)bromide, 33.1 mg of triphenylphosphine, 0.76 g of lithium bromide and 25 mg of sulfuric acid under 60 bar of carbon monoxide pressure at 120° C. for 12 hours in a 300 ml autoclave. The volatile constituents are subsequently removed in a high vacuum. The residue is taken up in saturated aqueous NaHCO₃ solution and washed with chloroform and ethyl acetate. The aqueous phase is adjusted to a pH of 2 using phosphoric acid and is extracted with ethyl acetate. The combined organic phases are dried over magnesium sulfate and the solvent is removed under reduced pressure. The product is recrystallized from a suitable solvent mixture.

### Example 21

2.8 g of cyclohexanecarbaldehyde and 1.5 g of acetamide were reacted using the general procedure II. 4.9 g of N-acetylcyclohexylglycine are found, corresponding to a yield of 99%. Selected NMR data:¹H-NMR (400 MHz, DMSO-d₆, 25° C.): δ=7.9 (d, 1H, NH), 4.1 (dd, 1H, α-CH), 1.8 (s, 3H, COCH₃).

### Example 22

2.2 g of pivalaldehyde and 1.5 g of acetamide were reacted using the general procedure II. 4.0 g of N-acetyl-tert-leucine are found, corresponding to a yield of 92%. Selected NMR data: ¹H-NMR (400 MHz, DMSO-d₆, 25° C.): δ=7.7 (d, 1H, NH), 3.9 (d, 1H, α-CH), 1.8 (s, 3H, COCH₃).

### Example 23

0.8 g of formaldehyde and 3.7 g of phthalimide were reacted using the general procedure II. 3.1 g of N-phthaloylglycine are found, corresponding to a yield of 60%. Selected NMR data: ¹H-NMR (400 MHz, DMSO-d₆, 25° C.): δ=4.3 (s, 2H, α-CH₂).

### Example 24

2.2 g of isovaleraldehyde and 2.2 g of methoxyacetamide were reacted using the general procedure II. 3.0 g of N-methoxyacetylleucine are found, corresponding to a yield of 59%. Selected NMR data: ¹H-NMR (400 MHz, DMSO-d₆, 25° C.): δ=7.9 (d, 1H, NH), 4.3 (dt, 1H, α-CH), 3.8 (s, 2H, —COCH₂—), 3.3 (s, 3H, —OCH₃).

### Example 25

2.8 g of cyclohexanecarbaldehyde and 2.2 g of methoxyacetamide were reacted using the general procedure II. 4.9 g of N-methoxyacetylcyclohexylglycine are found, corresponding to a yield of 85%. Selected NMR data: ¹H-NMR (400 MHz, DMSO-d₆, 25° C.): δ=7.6 (d, 1H, NH), 4.2 (dd, 1H, α-CH), 3.9 (s, 2H, —COCH₂—), 3.2 (s, 3H, —OCH₂).

### Example 26

2.2 g of isovaleraldehyde and 3.4 g of phenacetamide were reacted using the general procedure II. 5.1 g of N-phenacetylleucine are found, corresponding to a yield of 82%. Selected NMR data: ¹H-NMR (400 MHz, DMSO-d₆, 25° C.): δ=8.3 (d, 1H, NH), 4.2 (dt, 1H, α-CH), 3.5 (s, 2H, —COCH₂—).

### Example 27

2.7 g of benzaldehyde and 3.4 g of phenacetamide were reacted using the general procedure II. 4.4 g of N-phenacetylphenylglycine are found, corresponding to a yield of 65%. Selected NMR data: ¹H-NMR (400 MHz, DMSO-d₆, 25° C.): δ=8.8 (d, 1H, NH), 5.3 (d, 1H, α-CH), 3.6 (s, 2H, —COCH₂—).

### Example 28

2.8 g of cyclohexanecarbaldehyde and 1.2 g of formamide were reacted using the general procedure II. 1.1 g of N-formylcyclohexylglycine are found, corresponding to a yield of 25%. Selected NMR data: ¹H-NMR (400 MHz, DMSO-d₆, 25° C.): δ=8.2 (d, 1H, NH), 7.8 (s, 1H, —CHO), 4.1 (dd, 1H, α-CH).

1. A process for preparing N-acylglycine derivatives of the formula (III) 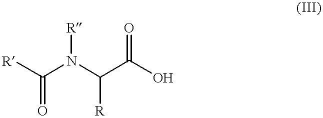
where
R is hydrogen, a carboxyl group, a saturated, straight-chain, branched or cyclic (C₁-C₁₀)alkyl radical, a monounsaturated or polyunsaturated, straight-chain, branched or cyclic (C₂-C₁₀)alkenyl radical, a (C₆-C₁₈)aryl radical, a (C₆-C₁₈)heteroaryl radical, a (C₁-C₁₀)alkyl-(C₆-C₁₈)aryl radical, a (C₁-C₁₀)alkyl-(C₆-C₁₈)heteroaryl radical or a monounsaturated or polyunsaturated (C₂-C₁₀)alkenyl-(C₆-C₁₈)aryl radical, where one or more radicals —CH₂— can be replaced by C═O or —O—,
R′ is hydrogen, a saturated, straight-chain, branched or cyclic (C₁-C₂₆)alkyl radical, a monounsaturated or polyunsaturated, straight-chain, branched or cyclic (C₂-C₂₄)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical
and
R″ is hydrogen, a saturated, straight-chain, branched or cyclic (C₁-C₂₆)alkyl radical, a monounsaturated or polyunsaturated, straight-chain, branched or cyclic (C₂-C₂₃)alkenyl radical, a (C₆-C₁₈)aryl radical, a (C₁-C₁₀)alkyl-(C₆-C₁₈)aryl radical or a monounsaturated or polyunsaturated (C₂-C₁₀)alkenyl-(C₆-C₁₈)aryl radical,
where R, R′ and R″ may be substituted,
which comprises carbonylating a carboxamide of the formula (II) 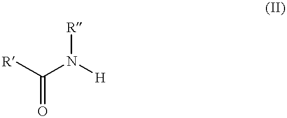
where R′ and R″ are as defined above, together with an aldehyde of the formula RCHO, where R is as defined above, in the presence of a solvent, a palladium compound, an ionic halide and an acid as catalyst at a temperature of 20-200° C. and a CO pressure of 1-150 bar.

2. The process as claimed in claim 1, wherein the carboxamide of the formula (II) is selected from the group consisting of the amides and N-methylamides of natural fatty acids, benzamide, phenylacetamide and 2-ethylhexanoic amide.

3. The process as claimed in claim 1, wherein R″ is hydrogen or (C₁-C₁₂)alkyl.

4. The process as claimed in claim 3, wherein R″ is methyl.

5. The process as claimed in any of the preceding claims, wherein the compounds of the formula (II) are used as mixtures as are obtainable from natural products.

6. The process as claimed in any of the preceding claims, wherein the aldehyde of the formula (I) is selected from the group consisting of formaldehyde, acetaldehyde, benzaldehyde, furfural, propionaldehyde, butyraldehyde, glyoxalic acid and isobutyraldehyde.

7. The process as claimed in any of the preceding claims, wherein the aldehyde is used in the form of its trimers or oligomers.

8. The process as claimed in claim 7, wherein formaldehyde is used in the form of paraformaldehyde.

9. The process as claimed in any of the preceding claims, wherein the aldehyde is used in an amount of from 70 to 200 mol %, based on the carboxamide.

10. The process as claimed in any of the preceding claims, wherein the palladium compound is selected from the group consisting of palladium(0) compounds, palladium(II) compounds and palladium-phosphine complexes.

11. The process as claimed in claim 10, wherein the palladium compound is selected from the group consisting of PdBr₂, PdCl₂, Pd(OAc)₂, Li₂PdBr₄, Li₂PdCl₄, and also the triphenylphosphine, tritolylphosphine, bis(diphenylphosphino)ethane, 1,4-bis(diphenylphosphino)butane and 1,3-bis(diphenylphosphino)propane complexes of palladium(II).

12. The process as claimed in claim 11, wherein the palladium compound used is bis(triphenylphosphine)palladium(II) chloride (PdCl₂[PPh₃]₂), bromide (PdBr₂[PPh₃]₂) or iodide (Pdl₂[PPh₃]₂).

13. The process as claimed in claim 10, wherein the phosphine used contains one or more chiral centers.

14. The process as claimed in any of claims 10 to 13, wherein the palladium compound, calculated as palladium metal, is used in an amount of from 0.0001 to 5 mol % based on the carboxamide.

15. The process as claimed in claim 1, wherein the ionic halide is selected from the group consisting of tetrabutylphosphonium bromide and iodide, ammonium, lithium, sodium and potassium bromide and ammonium, lithium, sodium and potassium iodide.

16. The process as claimed in claim 1, wherein the ionic halide is a bromide.

17. The process as claimed in claim 1, wherein the ionic halide is used in an amount of from 1 to 50 mol % based on the carboxamide.

18. The process as claimed in claim 1, wherein the acid is an organic or inorganic acid having a pKₐ<5 (relative to water).

19. The process as claimed in claim 18, wherein the acid is selected from the group consisting of sulfuric acid, trifluoroacetic acid, acetic acid, hexafluoropropanoic acid, p-toluenesulfonic acid, phosphoric acid and an ion-exchange resin having a pKₐ<5 (relative to water).

20. The process as claimed in either claim 18 or 19, wherein the acid is used in an amount of from 0.1 to 20 mol % based on the carboxamide.

21. The process as claimed in any of the preceding claims, wherein the solvent used contains product up to the saturation limit.

22. The process as claimed in any of the preceding claims, wherein the reaction is carried out at pressures of from 1 to 150 bar and at temperatures of from 20 to 200° C.

23. A process for preparing optically pure amino acids, which comprises converting the racemic N-acylglycine derivatives obtained by the process as claimed in any of claims 1 to 22 into the corresponding optically pure amino acids by means of stereoselective enzymatic hydrolysis.

24. The process as claimed in claim 23, wherein the stereoselective enzymatic hydrolysis is carried out using an enzyme selected from the group consisting of acylases, amidases and carboxypeptidases.
